(19) United States
(12) Patent Application Publication
(10) Pub. No.: US 2021/0179710 A1
(43) Pub. Date: Jun. 17, 2021

(54) COMBINATION IMMUNOTHERAPY AND CHEMOTHERAPY FOR THE TREATMENT OF A HEMATOLOGICAL MALIGNANCY

(71) Applicant: Actinium Pharmaceuticals, Inc., New York, NY (US)

(72) Inventor: Mark Berger, New York, NY (US)

(73) Assignee: Actinium Pharmaceuticals, Inc., New York, NY (US)

(21) Appl. No.: 16/954,105

(22) PCT Filed: Jan. 8, 2019
(86) PCT No.: PCT/US19/12647

§ 371 Date: Jun. 15, 2020

Related U.S. Application Data
(60) Provisional application No. 62/614,658, filed on Jan. 8, 2018.

(51) Int. Cl. C07K 16/28; A61K 31/7048; A61K 31/706; A61K 31/136; A61K 38/19; A61K 51/10; A61P 35/00

(52) U.S. Cl. CPC C07K 16/2803; A61K 31/7048; A61K 31/706; A61K 2039/505; A61K 38/193; A61K 51/1069; A61P 35/00; A61K 31/136

(57) ABSTRACT

Methods for treating a proliferative disease by administering a synergistic combination of an antibody against an epitope of CD33 (anti-CD33) and a combination of two or more individual chemotherapeutic agents is disclosed. The combination of chemotherapeutic agents may include cladribine, cytarabine, mitoxantrone, and filgrastim (CLAG-M), and the anti-CD33 may be a humanized monoclonal antibody against CD33 such as HuM195 conjugated with a radionuclide such as ¹³¹I or ²²⁵Ac. The proliferative disease or disorder may be a hematological disease such as multiple myeloma, acute myeloid leukemia, and/or myelodysplastic syndrome. The method may be particularly useful for treatment of relapsed or refractory acute myeloid leukemia.

### CROSS-REFERENCE TO RELATED APPLICATIONS

The present application is a 371 national entry of PCT/US2019/012647 filed Jan. 8, 2019 which claims the benefit under 35 U.S.C. § 119(e) of prior U.S. Provisional Application Ser. No. 62/614,658, filed Jan. 8, 2018, the contents of both are incorporated herein in their entirety.

### SEQUENCE LISTING

This application contains a Sequence Listing in computer readable form which is incorporated herein by reference. The sequence listing text file “SequenceListing17046_ST25” submitted via EFS in compliance with 37 CFR § 1.52(e)(5) and Rule 13ter.1(a) is identical to the sequence listing forming part of this international application.

### FIELD OF THE INVENTION

The presently disclosed invention relates to methods of treating a subject having a proliferative disorder by administration of an immunotherapeutic agent against an epitope of CD33 and a chemotherapy, and more specifically, the invention relates to administration of an anti-CD33 antibody and CLAG-M chemotherapy for the treatment of a hematological disease or disorder.

### BACKGROUND OF THE INVENTION

Hematological malignancies have historically been treated using high dose chemotherapies and/or radiation. Current treatment protocols generally include a combination of chemotherapeutic agents such as vincristine, carmustine, cytarabine, melphalan, cyclophosphamide, daunorubicin, and steroidal agents such as prednisone or dexamethasone. Such treatment, however, yields low remission rates and poor overall survival rates for hematological diseases, such as acute myeloid leukemia (AML).

For example, recent advances using high dose chemotherapy followed by autologous bone marrow or peripheral blood mononuclear cell transplantation has increased the complete remission rate and remission duration for multiple myeloma (MM). Yet no evidence for a cure has been obtained. The efficacy of these available chemotherapeutic treatment regimens for MM is limited because MM exhibits a low proliferation rate, develops multi-drug resistance, and has a high resistance to apoptosis. Approximately 1% of all cancers, and slightly more than 10% of all hematologic malignancies, can be attributed to MM. The vast majority of MM patients will relapse within a few years, and in most cases the relapsed patients do not respond well to salvage therapy. For more than 90% of MM patients, the disease eventually becomes chemoresistant.

Relapsed/Refractory acute myeloid leukemia (RR-AML) in adults is a particularly difficult therapeutic challenge. Its treatment response is variable with the use of different salvage chemotherapy regimens aimed at achieving disease remission in order to proceed to stem cell transplantation. While there is no universally accepted regimen to date, various regimens such as CLAG-M (cladribine, cytarabine, mitoxantrone, and filgrastim), FLAG (fludarabine, cytarabine, idarubicin, and filgrastim), or MEC (mitoxantrone, etoposide, and cytarabine) have been used. CLAG-M has been found to produce a morphological complete response rate of 58% in prospective clinical trials for the treatment of RR-AML.

A recent retrospective study compared two commonly used regimens in RR-AML: CLAG (cladribine, cytarabine, and filgrastim) and MEC (mitoxantrone, etoposide & cytarabine). See Price et al., Leukemia Research, vol. 35, issue 3, pages 301-304. The complete response rate was found to be 37.9% for CLAG (n=97) and 23.8% for MEC (n=65) (P=0.048), with a median overall survival of 7.3 and 4.5 months, respectively (P=0.05). In primary refractory disease, the complete response rate was 45.5% for CLAG and 22.2% for MEC (P=0.09), with a median overall survival of 11 and 4.5 months, respectively (P=0.07). In patients with relapsed AML, the complete response rate was 36.8% with CLAG and 25.9% with MEC (P=0.35) and the median overall survival was 6.7 and 6.7 months, respectively (P=0.87). The combination of the purine nucleoside analogue (cladribine) with cytarabine increases the intracellular accumulation of Ara-C-5′ triphosphate (ara-C TP) that causes cytotoxicity in leukemic blasts. Addition of granulocyte-colony stimulating factor (G-CSF) further improves the effects of a purine nucleoside analogue in combination with Ara-C by activating the leukemic cells and making them susceptible to chemotherapy's effect.

Phenotypic changes distinguishing cancerous cells from normal cells derived from the same tissue, or cell type, are often associated with one or more changes in the expression of specific gene products, including the loss of normal cell surface components or the gain of others (i.e., antigens not detectable in corresponding normal, non-cancerous cells). The antigens which are expressed by cancerous cells (i.e., in or on the cancerous cells), but not by normal cells, or which are expressed by cancerous cells at levels substantially above those found in normal cells, are often referred to as “tumor-specific antigens” or “tumor-associated antigens.” Such tumor-specific antigens may serve as markers for a tumor phenotype, and have been used as targets for cancer immunotherapies.

As such, an important strategy that could enhance the efficacy of killing leukemic blasts when added to chemotherapy are monoclonal antibodies directed against markers expressed in leukemic or malignant cells. An object of the presently disclosed invention is to provide improved methods for the treatment of proliferative disorders such as hematological diseases.

### SUMMARY OF THE INVENTION

The presently disclosed invention is based on the discovery that administration of a combination comprising at least one immunotherapeutic agent, such as a monoclonal antibody against CD33, and a combination of two or more chemotherapeutic agents, such as the combination of cladribine, cytarabine, mitoxantrone, and granulocyte colony stimulating factor or filgrastim (CLAG-M), has therapeutic synergy and/or improves efficacy in the treatment of a proliferative disease over use of the immunotherapeutic agent alone, or the chemotherapeutic agent(s) alone.

Accordingly, the presently disclosed invention relates to methods for treating a subject having a proliferative disorder, such as multiple myeloma, acute myeloid leukemia, myelodysplastic syndrome, or myeloproliferative neoplasm, and especially for the treatment relapsed or refractory states of these diseases. The methods generally comprise administering an effective amount of an immunotherapy directed to CD33 and an effective amount of a combination of two or more chemotherapeutic agents. According to certain aspects, three or more chemotherapeutic agents may be used in the disclosed methods.

The presently disclosed invention also relates to methods for inhibiting growth and/or proliferation of a cell expressing CD33, and methods of treating a disease or disorder involving cells expressing CD33. Both methods comprise administering to a subject an effective amount of an immunotherapy directed to CD33 and an effective amount of a combination of two or more chemotherapeutic agents, or an effective amount of three or more chemotherapeutic agents.

According to certain aspects of the presently disclosed invention, the combination of two or more chemotherapeutic agents, and the combination of three or more chemotherapeutic agents, may comprise a combination of individual chemotherapeutic agents selected from cladribine, cytarabine, mitoxantrone, and granulocyte colony stimulating factor or filgrastim. The chemotherapeutic agents may be delivered together and/or individually during a treatment period, such as on a daily or hourly schedule specific for each chemotherapeutic agent.

According to certain aspects of the presently disclosed invention, the immunotherapeutic agent may comprise an immunotherapy directed to CD33, such as an anti-CD33 antibody conjugated with a radiolabel. The anti-CD33 antibody may be administered on any one or more of days 1 to 30 of the treatment period. When more than one dose of the anti-CD33 antibody is administered, the doses may be the same or may vary.

The objects of the presently disclosed invention will be realized and attained by means of the combinations specifically outlined in the appended claims. The foregoing general description and the following detailed description and examples of this invention are provided to illustrate various aspects of the presently disclosed invention, and by no means are to be viewed as limiting any of the described embodiments.

### BRIEF DESCRIPTION OF THE DRAWINGS

FIG. 1 provides the amino acid sequence of human CD33 as shown in GenBank accession number NP_001763.

### DETAILED DESCRIPTION OF THE INVENTION

The presently disclosed invention is related to methods for treating a proliferative disease or disorder by administering an effective amount of an immunotherapy such as a CD33 targeting agent, and an effective amount of a chemotherapy such as CLAG-M. Each therapy regime (i.e., immunotherapy and chemotherapy) may be administered according to a specific dosing schedule, wherein the method provides for administration of each therapy according to the dosing schedule sequentially (the antibody dosing schedule is completed before the chemotherapy dosing schedule is started, or vice versa) or simultaneously.

### Definitions

Throughout this description and in the appended claims, use of the singular includes the plural and plural encompasses singular, unless specifically stated otherwise. For example, although reference is made herein to “an” antibody, “a” radionuclide, and “the” chemotherapeutic, one or more of any of these components and/or any other components described herein may be used.

The word “comprising” and forms of the word “comprising”, as used in this description and in the claims, does not limit the presently disclosed invention to exclude any variants or additions. Additionally, although the presently disclosed invention has been described in terms of “comprising”, the processes, materials, and compositions detailed herein may also be described as consisting essentially of or consisting of. For example, while certain aspects of the invention have been described in terms of a method comprising administering an immunotherapy against CD33 and a chemotherapeutic treatment regime, such as CLAG-M, a method “consisting essentially of” or “consisting of” administering the immunotherapy against CD33 and a chemotherapeutic treatment regime is also within the present scope. In this context, “consisting essentially of” means that any additional components will not materially affect the efficacy of the method.

Moreover, other than in the examples, or where otherwise indicated, all numbers expressing, for example, quantities of ingredients used in the specification are to be understood as being modified in all instances by the term “about”. Accordingly, unless indicated to the contrary, the numerical parameters set forth in the following specification are approximations that may vary depending upon the desired properties to be obtained by the presently disclosed invention. At the very least, and not as an attempt to limit the application of the doctrine of equivalents to the scope of the claims, each numerical parameter should at least be construed in light of the number of reported significant digits and by applying ordinary rounding techniques. Thus, the term “about” when used before a numerical designation, e.g., temperature, time, amount, and concentration, including a range, indicates approximations which may vary by ±10%, ±5%, or ±1%.

As used herein, “administer”, with respect to a targeting agent such as an antibody, antibody fragment, Fab fragment, or aptamer, means to deliver the agent to a subject's body via any known method suitable for antibody delivery. Specific modes of administration include, without limitation, intravenous, transdermal, subcutaneous, intraperitoneal, intrathecal and intra-tumoral administration. Exemplary administration methods for antibodies may be as substantially described in International Publication No. WO 2016/187514, incorporated by reference herein. For example, according to certain aspects, the targeting agent may be administered as a patient specific therapeutic composition which may be included in a single dose container, the total volume of which may be administered to a patient in a single treatment session. The composition may include a monoclonal antibody or antibody fragment and a pharmaceutically acceptable carrier, wherein a dose of an effector molecule (e.g., radionuclide) of the monoclonal antibody and a total protein amount of the monoclonal antibody may depend on at least one patient specific parameter. Patient specific parameters include, but are not limited to, a patient weight, a patient age, a patient height, a patient gender, a patient medical condition, and a patient medical history.

In addition, in this invention, antibodies or antibody fragments can be formulated using one or more routinely used pharmaceutically acceptable carriers. Such carriers are well known to those skilled in the art. For example, injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's). An exemplary formulation may be as substantially described in International Publication No. WO 2017/155937, incorporated by reference herein. For example, according to certain aspects, the formulation may comprise 0.5% to 5.0% (w/v) of an excipient selected from the group consisting of ascorbic acid, polyvinylpyrrolidone (PVP), human serum albumin (HSA), a water-soluble salt of HSA, and mixtures thereof. Certain formulations may comprise 0.5-5% ascorbic acid; 0.5-4% polyvinylpyrrolidone (PVP); and the monoclonal antibody in 50 mM PBS buffer, pH 7.

As used herein, the term “antibody” includes, without limitation, (a) an immunoglobulin molecule comprising two heavy chains and two light chains and which recognizes an antigen; (b) polyclonal and monoclonal immunoglobulin molecules; (c) monovalent and divalent fragments thereof (e.g., di-Fab), and (d) bi-specific forms thereof. Immunoglobulin molecules may derive from any of the commonly known classes, including but not limited to IgA, secretory IgA, IgG and IgM. IgG subclasses are also well known to those in the art and include, but are not limited to, human IgG1, IgG2, IgG3 and IgG4. Antibodies can be both naturally occurring and non-naturally occurring (e.g., IgG-Fc-silent). Furthermore, antibodies include chimeric antibodies, wholly synthetic antibodies, single chain antibodies, and fragments thereof. Antibodies may be human, humanized or nonhuman.

As used herein, “Immunoreactivity” refers to a measure of the ability of an immunoglobulin to recognize and bind to a specific antigen. “Specific binding” or “specifically binds” or “binds” refers to an antibody binding to an antigen or an epitope within the antigen with greater affinity than for other antigens. Typically, the antibody binds to the antigen or the epitope within the antigen with an equilibrium dissociation constant (K_{D}) of about 1×10⁻⁸ M or less, for example about 1×10⁻⁹ M or less, about 1×10⁻¹⁰ M or less, about 1×10⁻¹¹ M or less, or about 1×10⁻¹² M or less, typically with the K_{D} that is at least one hundred fold less than its K_{D} for binding to a nonspecific antigen (e.g., BSA, casein). The dissociation constant may be measured using standard procedures. Antibodies that specifically bind to the antigen or the epitope within the antigen may, however, have cross-reactivity to other related antigens, for example to the same antigen from other species (homologs), such as human or monkey, for example Macaca fascicularis (cynomolgus, cyno), Pan troglodytes (chimpanzee, chimp) or Callithrix jacchus (common marmoset, marmoset).

As used herein, an “anti-CD33 targeting agent” is an antibody, antibody fragment, peptide, Fab fragment, or aptamer that binds to any available epitope of CD33. According to certain aspects, the anti-CD33 targeting agent is a humanized antibody against CD33, such as lintuzumab (HuM195), gemtuzumab, or vadastuximab. According to certain aspects, the anti-CD33 targeting agent binds to the epitope recognized by the monoclonal antibody “lintuzumab” or “HuM195.” HuM195 is known, as are methods of making it.

An “epitope” refers to the target molecule site (e.g., at least a portion of an antigen) that is capable of being recognized by, and bound by, a targeting agent such as an antibody, antibody fragment, Fab fragment, or aptamer. For a protein antigen, for example, this may refer to the region of the protein (i.e., amino acids, and particularly their side chains) that is bound by the antibody. Overlapping epitopes include at least one to five common amino acid residues. Methods of identifying epitopes of antibodies are known to those skilled in the art and include, for example, those described in Antibodies, A Laboratory Manual, Cold Spring Harbor Laboratory, Ed Harlow and David Lane (1988).

As used herein, “cancer” includes, without limitation, a solid cancer (e.g., a tumor) and a hematologic malignancy.

A “hematologic disease” or “hematological disorder” may be taken to refer to at least a blood cancer. Such cancers originate in blood-forming tissue, such as the bone marrow or other cells of the immune system. A hematologic disease or disorder includes, without limitation, leukemias (such as acute myeloid leukemia (AML), acute promyelocytic leukemia, acute lymphoblastic leukemia (ALL), acute mixed lineage leukemia, chronic myeloid leukemia (CML), chronic lymphocytic leukemia (CLL), hairy cell leukemia and large granular lymphocytic leukemia), myelodysplastic syndrome (MDS), myeloproliferative disorders (polycythemia vera, essential thrombocytosis, primary myelofibrosis and chronic myeloid leukemia), lymphomas, multiple myeloma, MGUS and similar disorders, Hodgkin's lymphoma (HL), non-Hodgkin lymphoma (NHL), primary mediastinal large B-cell lymphoma, diffuse large B-cell lymphoma, follicular lymphoma, transformed follicular lymphoma, splenic marginal zone lymphoma, lymphocytic lymphoma, T-cell lymphoma, and other B-cell malignancies.

“Solid cancers” include, without limitation, bone cancer, pancreatic cancer, skin cancer, cancer of the head or neck, cutaneous or intraocular malignant melanoma, uterine cancer, ovarian cancer, prostate cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, pediatric tumors, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, environmentally-induced cancers including those induced by asbestos.

“Antibody-dependent cellular cytotoxicity”, “antibody-dependent cell-mediated cytotoxicity” or “ADCC” is a mechanism for inducing cell death that depends upon the interaction of antibody-coated target cells with effector cells possessing lytic activity, such as natural killer (NK) cells, monocytes, macrophages and neutrophils via Fc gamma receptors (FcγR) expressed on effector cells. For example, NK cells express FcγRIIIa, whereas monocytes express FcγRI, FcγRII and FcvRIIIa. Death of the antibody-coated target cell, such as CD38-expressing cells, occurs as a result of effector cell activity through the secretion of membrane pore-forming proteins and proteases.

“Complement-dependent cytotoxicity”, or “CDC”, refers to a mechanism for inducing cell death in which an Fc effector domain of a target-bound antibody binds and activates complement component C1q, which in turn activates the complement cascade leading to target cell death. Activation of complement may also result in deposition of complement components on the target cell surface that facilitate ADCC by binding complement receptors (e.g., CR3) on leukocytes.

“Apoptosis” refers to a mechanism of programmed cell death wherein antibody binding to the target cell disrupts integral cell signaling pathways and results in cell self-destruction.

According to certain aspects, the anti-CD33 targeting agent may be labelled with a radioisotope. Methods of labeling proteins such as antibodies with radioisotopes, such as the radioisotopes ¹³¹I or ²²⁵Ac, are known. These methods are described, for example, in International Publication No. WO 2017/155937 or International Application No. PCT/US18/44531. For example, according to certain aspects, the anti-CD33 targeting agent may be labelled by (a) reacting the targeting agent with a chelant in a buffered solution, (b) reacting the chelated targeting agent with a radionuclide in a buffered solution, (c) quenching the reaction by the addition of a quenching chelate (e.g. diethylenetriaminepentaacetic acid (DTPA)), and (d) purifying the radiolabeled-chelated targeting agent. Exemplary chelators include compounds having the dual functionality of sequestering metal ions plus the ability to covalently bind a biological carrier such as an antibody. Exemplary chelators include, but are not limited to compounds such as S-2-(4-Isothiocyanatobenzyl)-1,4,7,10 tetraazacyclododecanetetraacetic acid (p-SCN-Bn-DOTA), diethylene triamine pentaacetic acid (DTPA); ethylene diamine tetraacetic acid (EDTA); 1,4,7,10-tetra-azacyclododecane-N,N′,N″,N′″-tetraacetic acid (DOTA); p-isothiocyanatobenzyl-1,4,7,10-tetra-azacyclododecane-1,4,7,10-tetraacetic acid (p-SCN-Bz-DOTA); 1,4,7,10-tetra-azacyclododecane-N,N′,N″-triacetic acid (DO3A); 1,4,7,10-tetra-azacyclododecane-1,4,7,10-tetrakis(2-propionic acid) (DOTMA); 3,6,9-triaza-12-oxa-3,6,9-tricarboxymethylene-10-carboxy-13-phenyl-tridecanoic acid (“B-19036”); 1,4,7-triazacyclononane-N,N′,N″-triacetic acid (NOTA); 1,4,8,11-tetra-azacyclotetradecane-N,N′,N″,N′″-tetraacetic acid (TETA); triethylene tetraamine hexaacetic acid (TTHA); trans-1,2-diaminohexane tetraacetic acid (CYDTA); 1,4,7,10-tetra-azacyclododecane-1-(2-hydroxypropyl)-4,7,10-triacetic acid (HP-DO3A); trans-cyclohexane-diamine tetraacetic acid (CDTA); trans(1,2)-cyclohexane diethylene triamine pentaacetic acid (CDTPA); 1-oxa-4,7,10-triazacyclododecane-N,N′,N″-triacetic acid (OTTA); 1,4,7,10-tetra-azacyclododecane-1,4,7,10-tetrakis(3-(4-carboxyl)-butanoic acid); 1,4,7,10-tetra-azacyclododecane-1,4,7,10-tetrakis(acetic acid-methyl amide); 1,4,7,10-tetra-azacyclododecane-1,4,7,10-tetrakis(methylene phosphonic acid); and derivatives thereof.

As used herein, a “radioisotope” and “radionuclide” may be used interchangeably, and can an alpha-emitting isotope, a beta-emitting isotope, and/or a gamma-emitting isotope. Examples of radioisotopes include the following: ¹³¹I, ¹²⁵I, ¹²³I, ⁹⁰Y, ¹⁷⁷Lu, ¹⁸⁶Re, ¹⁸⁸Re, ⁸⁹Sr, ¹⁵³Sm, ³²P, ²²⁵Ac, ²¹³Bi, ²¹³Po, ²¹¹At, ²¹²Bi, ²²³Bi, ²²³Ra, ²²⁷Th, ¹⁴⁹Tb, ¹³⁷Cs, ²¹²Pb and ¹⁰³Pd.

According to certain aspects, the anti-CD33 targeting agent may be an antibody radiolabeled with ¹³¹I (“¹³¹I-labeled”), and the effective amount may be below, for example, 1200 mCi (i.e., where the amount of ¹³¹I administered to the subject delivers a total body radiation dose of below 1200 mCi). According to certain aspects, when the antibody is ¹³¹I-labeled, the effective amount may be below 1000 mCi, below 750 mCi, below 500 mCi, below 250 mCi, below 200 mCi, below 150 mCi, below 100 mCi, below 50 mCi, below 40 mCi, below 30 mCi, below 20 mCi or below 10 mCi.

According to certain aspects, the effective amount of ¹³¹I-labeled antibody is from 10 mCi to 200 mCi. Examples of effective amounts include, without limitation, from 50 mCi to 100 mCi, from 50 mCi to 150 mCi, from 50 mCi to 200 mCi, from 60 mCi to 140 mCi, from 70 mCi to 130 mCi, from 80 mCi to 120 mCi, from 90 mCi to 110 mCi, from 100 mCi to 150 mCi, 50 mCi, 60 mCi, 70 mCi, 80 mCi, 90 mCi, 100 mCi, 110 mCi, 120 mCi, 130 mCi, 140 mCi, 150 mCi, or 200 mCi.

According to certain aspects, the effective amount of ¹³¹I-labeled antibody is from 200 mCi to 1200 mCi. Examples of effective amounts include, without limitation, from 200 mCi to 300 mCi, from 200 mCi to 400 mCi, from 200 mCi to 500 mCi, from 200 mCi to 600 mCi, from 200 mCi to 700 mCi, from 200 mCi to 800 mCi, from 200 mCi to 900 mCi, from 200 mCi to 1000 mCi, from 200 mCi to 1100 mCi, from 300 mCi to 1200 mCi, from 400 mCi to 1200 mCi, from 500 mCi to 1200 mCi, from 600 mCi to 1200 mCi, from 700 mCi to 1200 mCi, from 800 mCi to 1200 mCi, from 900 mCi to 1200 mCi, from 1000 mCi to 1200 mCi, 50 mCi, 100 mCi, 150 mCi, 200 mCi, 300 mCi, 400 mCi, 500 mCi, 600 mCi, 700 mCi, 800 mCi, 900 mCi, 1000 mCi, or 1100 mCi.

According to certain aspects, anti-CD33 targeting agent may be an antibody radiolabeled with ²²⁵Ac (“²²⁵Ac-labeled”), and the effective amount may be below, for example, 5.0 μCi/kg (i.e., where the amount of ²²⁵Ac administered to the subject delivers a radiation dose of below 5.0 μCi per kilogram of subject's body weight). According to certain aspects, when the antibody is ²²⁵Ac-labeled, the effective amount is below 4.5 μCi/kg, 4.0 μCi/kg, 3.5 μCi/kg, 3.0 μCi/kg, 2.5 μCi/kg, 2.0 μCi/kg, 1.5 μCi/kg, 1.0 μCi/kg, 0.9 μCi/kg, 0.8 μCi/kg, 0.7 μCi/kg, 0.6 μCi/kg, 0.5 μCi/kg, 0.4 μCi/kg, 0.3 μCi/kg, 0.2 μCi/kg, 0.1 μCi/kg or 0.05 μCi/kg.

According to certain aspects, when the antibody is ²²⁵Ac-labeled, the effective amount is from 0.05 μCi/kg to 0.1 μCi/kg, from 0.1 μCi/kg to 0.2 μCi/kg, from 0.2 μCi/kg to 0.3 μCi/kg, from 0.3 μCi/kg to 0.4 μCi/kg, from 0.4 μCi/kg to 0.5 μCi/kg, from 0.5 μCi/kg to 0.6 μCi/kg, from 0.6 μCi/kg to 0.7 μCi/kg, from 0.7 μCi/kg to 0.8 μCi/kg, from 0.8 μCi/kg to 0.9 μCi/kg, from 0.9 μCi/kg to 1.0 μCi/kg, from 1.0 μCi/kg to 1.5 μCi/kg, from 1.5 μCi/kg to 2.0 μCi/kg, from 2.0 μCi/kg to 2.5 μCi/kg, from 2.5 μCi/kg to 3.0 μCi/kg, from 3.0 μCi/kg to 3.5 μCi/kg, from 3.5 μCi/kg to 4.0 μCi/kg, from 4.0 μCi/kg to 4.5 μCi/kg, or from 4.5 μCi/kg to 5.0 μCi/kg.

According to certain aspects, when the antibody is ²²⁵Ac-labeled, the effective amount is 0.05 μCi/kg, 0.1 μCi/kg, 0.2 μCi/kg, 0.3 μCi/kg, 0.4 μCi/kg, 0.5 μCi/kg, 0.6 μCi/kg, 0.7 μCi/kg, 0.8 μCi/kg, 0.9 μCi/kg, 1.0 μCi/kg, 1.5 μCi/kg, 2.0 μCi/kg, 2.5 μCi/kg, 3.0 μCi/kg, 3.5 μCi/kg, 4.0 μCi/kg or 4.5 μCi/kg.

According to certain aspects of the presently disclosed invention, when the anti-CD33 targeting agent is labeled with a radioisotope, the majority of the targeting agent (antibody, antibody fragment, etc.) administered to a subject typically consists of non-labeled targeting agent, with the minority being the labeled targeting agent. The ratio of labeled to non-labeled targeting agent can be adjusted using known methods. Thus, accordingly to certain aspects of the presently disclosed invention, the anti-CD33 targeting agent may be provided in a total protein amount of up to 100 mg, such as up to 60 mg, such as 5 mg to 45 mg, or a total protein amount of between 0.01 mg/kg patient weight to 16.0 mg/kg patient weight, such as between 0.01 mg/kg patient weight to 10.0 mg/kg, or between 0.05 mg/kg patient weight to 5.0 mg/kg, or between 0.01 mg/kg patient weight to 1.0 mg/kg, or between 0.01 mg/kg patient weight to 0.6 mg/kg patient weight, or 0.01 mg/kg patient weight, 0.015 mg/kg patient weight, 0.02 mg/kg patient weight, or 0.04 mg/kg patient weight, or 0.06 mg/kg patient weight. According to certain aspects of the presently disclosed invention, the effective dose of the anti-CD33 antibody may be a dose of less than 10 mg/m², such as about 6 mg/m², or 3 mg/m², or even 2 mg/m².

According to certain aspects of the presently disclosed invention, when the anti-CD33 targeting agent is radiolabeled, the radiolabeled anti-CD33 targeting agent may comprise a labeled fraction and an unlabeled fraction, wherein the ratio of labeled:unlabeled may be from about 0.01:10 to 1:1, such as 0.1:10 to 1:1 labeled:unlabeled. Moreover, the radiolabeled anti-CD33 targeting agent may be provided as a single dose composition tailored to a specific patient, wherein the amount of labeled and unlabeled anti-CD33 targeting agent in the composition may depend on at least a patient weight, age, and/or disease state or health status, such as detailed in International Publication No. WO 2016/187514.

As used herein, the term “subject” includes, without limitation, a mammal such as a human, a non-human primate, a dog, a cat, a horse, a sheep, a goat, a cow, a rabbit, a pig, a rat and a mouse. Where the subject is human, the subject can be of any age. For example, the subject can be 60 years or older, 65 or older, 70 or older, 75 or older, 80 or older, 85 or older, or 90 or older. Alternatively, the subject can be 50 years or younger, 45 or younger, 40 or younger, 35 or younger, 30 or younger, 25 or younger, or 20 or younger. For a human subject afflicted with cancer, the subject can be newly diagnosed, or relapsed and/or refractory, or in remission.

As used herein, “treating” a subject afflicted with a cancer shall include, without limitation, (i) slowing, stopping or reversing the cancer's progression, (ii) slowing, stopping or reversing the progression of the cancer's symptoms, (iii) reducing the likelihood of the cancer's recurrence, and/or (iv) reducing the likelihood that the cancer's symptoms will recur. According to certain preferred aspects, treating a subject afflicted with a cancer means (i) reversing the cancer's progression, ideally to the point of eliminating the cancer, and/or (ii) reversing the progression of the cancer's symptoms, ideally to the point of eliminating the symptoms, and/or (iii) reducing or eliminating the likelihood of relapse (i.e., consolidation, which ideally results in the destruction of any remaining cancer cells).

“Chemotherapeutic”, in the context of this invention, shall mean a chemical compound which inhibits or kills growing cells and which can be used or is approved for use in the treatment of cancer. Exemplary chemotherapeutic agents include alkylating agents, plant alkaloids, antimetabolites, anti-tumor antibiotics, topoisomerase inhibitors, mitotic inhibitors, corticosteroids, and cytostatic agents which prevent, disturb, disrupt or delay cell division at the level of nuclear division or cell plasma division.

“Therapeutically effective amount” or “effective amount” refers to an amount effective, at dosages and for periods of time necessary, to achieve a desired therapeutic result. A therapeutically effective amount may vary according to factors such as the disease state, age, sex, and weight of the individual, and the ability of a therapeutic or a combination of therapeutics to elicit a desired response in the individual. Exemplary indicators of an effective therapeutic or combination of therapeutics include, for example, improved well-being of the patient, reduction in a tumor burden, arrested or slowed growth of a tumor, and/or absence of metastasis of cancer cells to other locations in the body. According to certain aspects, “therapeutically effective amount” or “effective amount” refers to an amount of the anti-CD33 targeting agent that may deplete or cause a reduction in the overall number of cells expressing CD33, or may inhibit growth of cells expressing CD33.

As used herein, “depleting”, with respect to cells expressing CD33, shall mean to lower the population of at least one type of cells that express of overexpress CD33 (e.g., at least one type of the subject's peripheral blood lymphocytes or at least one type of the subject's bone marrow lymphocytes). According to certain aspects of this invention, a subject's lymphocyte decrease is determined by measuring the subject's peripheral blood lymphocyte level. As such, and by way of example, a subject's lymphocyte population is depleted if the population of at least one type of the subject's peripheral blood lymphocytes is lowered, such as by at least 10%, 20%, 30%, 40%, 50%, 60%, 70%, 80%, 90% or 99%.

“Inhibits growth” refers to a measurable decrease or delay in the growth of a malignant cell or tissue (e.g., tumor) in vitro or in vivo when contacted with a therapeutic or a combination of therapeutics or drugs, when compared to the decrease or delay in the growth of the same cells or tissue in the absence of the therapeutic or the combination of therapeutic drugs. Inhibition of growth of a malignant cell or tissue in vitro or in vivo may be at least about 10%, 20%, 30%, 40%, 50%, 60%, 70%, 80%, 90%, 95%, 96%, 97%, 98%, 99%, or 100%.

“Synergistic combinations,” as used herein, are combinations of monotherapies that may provide a therapeutic effect that is comparable to the effectiveness of a monotherapy, while reducing adverse side effects, e.g. damage to non-targeted tissues, immune status, and other clinical indicia. Alternatively, synergistic combinations may provide for an improved effectiveness, which may be measured by total tumor cell number, length of time to relapse, and other indicia of patient health.

Synergistic combinations of the presently disclosed invention combine an immunotherapy against CD33, i.e., an agent that is targeted to inhibit or block CD33 function, such as a monoclonal antibody against CD33, and an agent that is targeted to reduce proliferation of certain cell types, such as a chemotherapeutic agents or combination of chemotherapeutic agents.

Throughout this application, various publications are cited. The disclosure of these publications is hereby incorporated by reference into this application to describe more fully the state of the art to which this invention pertains.

Unless otherwise defined, all technical and scientific terms used herein have the same meaning as commonly understood by one of ordinary skill in the art to which the presently disclosed invention belongs. Although methods and materials similar or equivalent to those described herein can be used in the practice or testing described herein, suitable methods and materials are described below.

### ASPECTS OF THE INVENTION

Overexpression of CD33 is commonly found in many hematological malignancies, including AML, CML, and MDS. In AML, 85-90% of patients express CD33, which has led to the development of targeted therapies, such as gemtuzumab-ozogamicin (Mylotarg). Approximately 96% of MDS patients express CD33 on their myeloblasts (Sanford et al., “CD33 is frequently expressed in cases of myelodysplastic syndrome and chronic myelomonocytic leukemia with elevated blast count,” 2016, Leukemia & Lymphoma, vol. 57(8):1965-1968). In another study, MDS patients demonstrated approximately twice as many CD33 molecules per bone marrow cell as the control samples (Jilani, et al., “Differences in CD33 intensity between various myeloid neoplasms,” 2002, Am J Clin Pathol 2002, vol. 118:560-566). The CD33 antigen is expressed on virtually all cases of CML. Moreover, patients older than 60 years have a poor prognosis with only 10% to 15% of 4-year disease-free survival for AML. This high relapse rate for AML patients and the poor prognosis for older patients highlight the urgent need for novel therapeutics preferentially targeting CD33⁺ cells.

Accordingly, the methods disclosed herein include administration of an immunotherapy against CD33. The methods may be used to treat a proliferative disorder such as a hematological disease or disorder, and/or may be used to inhibit growth and/or proliferation of a cell expressing CD33, and/or may also be used to treat a disease or disorder involving cells expressing or overexpressing CD33. Moreover, the method may treat a relapsed/refractory hematological disease or disorder, wherein the hematological disease or disorder is selected from multiple myeloma, acute myeloid leukemia, myelodysplastic syndrome, and myeloproliferative neoplasm.

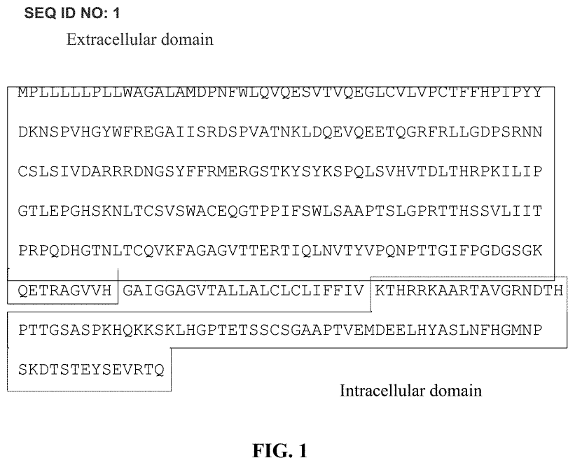

Human CD33 has an amino acid sequence shown in GenBank accession number NP_001763 and in SEQ ID NO: 1 (FIG. 1). CD33 is a 67 Kd type I transmembrane receptor glycoprotein that may function as a sialic acid-dependent cell adhesion molecule. CD33 has a long N-terminal extracellular domain, a helical transmembrane domain, and a short C-terminal cytoplasmic domain. Expressed on early myeloid progenitor and myeloid leukemic (e.g., acute myelogenous leukemia, AML) cells, CD33 is not expressed on stem cells.

With reference to FIG. 1, amino acid residues 1-259 represent the extracellular domain, amino acids 260-282 represent the helical transmembrane domain, and amino acids 283-364 represent the cytosolic domain (intracellular). There are at least three known single nucleotide polymorphisms (“SNPs”) in the extracellular domain of CD33 (i.e., W22R, R69G, S128N). Therefore, the extracellular domain of Homo sapiens CD33 can have the amino acid sequence of SEQ ID NO:1 with any one or more of these SNPs.

Recent studies suggest a role for CD33 in the modulation of inflammatory and immune responses through a dampening effect on tyrosine kinase-driven signaling pathways. For example, in vitro studies have demonstrated that CD33 constitutively suppresses the production of pro-inflammatory cytokines such as IL-1β, TNF-α, and IL-8 by human monocytes in a sialic acid ligand-dependent and SOCS3-dependent manner. Conversely, reduction of cell surface CD33 or interruption of sialic acid binding can increase p38 mitogen-activated protein kinase (MAPK) activity and enhance cytokine secretion as well as cytokine-induced cellular proliferation.

Antibodies against CD33, such as lintuzumab (HuM195), gemtuzumab, and vadastuximab have been, and are currently being evaluated in the clinic for their efficacy to treat hematological malignancies and plasma cell disorders, including acute myeloid leukemia (AML). Each antibody has been found to bind to a different portion of the extracellular region of CD33, and each demonstrates different clinical responses (e.g., anti-tumor effects). Gemtuzumab is available from Pfizer as Mylotarg™, and vadastuximab is available from Seattle Genetics as Vadastuximab talirine.

For example, the antibody lintuzumab (HuM195) has demonstrated anti-leukemic effects in the treatment of AML. HuM195 is a recombinant humanized anti-CD33 monoclonal antibody originally produced by Protein Design Labs, Inc. (Fremont, Calif.). M195 is a monoclonal IgG2a antibody that binds CD33. M195 is derived from a mouse immunized with live human leukemic myeloblasts. HuM195 was constructed by grafting complementarity-determining regions of M195 into a human IgG framework and backbone. HuM195 induced antibody-dependent cell-mediated cytotoxicity using human peripheral blood mononuclear cells as effectors. Four clinical trials have investigated native (i.e., unconjugated) HuM195 alone in patients with relapsed or refractory AML and CML. Fever, chills, and nausea were the most common toxicities. Human anti-human antibody responses were not seen. Beneficial biologic activity in terms of reduction in marrow blast cells was seen in some patients. Those who benefited the most had fewer blasts at the beginning of therapy, suggesting that HuM195 may be more effective in the treatment of minimal residual or cytoreduced disease.

Proposed methods by which these antibodies eliminate CD33-positive cells include antibody-dependent cellular cytotoxicity (ADCC), complement-dependent cytotoxicity (CDC), and apoptosis.

To assess ADCC activity of an antibody that binds to a specific antigen, such as an antibody against CD33, the antibody may be added to antigen-expressing cells in combination with immune effector cells, which may be activated by the antigen-antibody complexes resulting in cytolysis of the antigen-expressing cells, respectively. Cytolysis is generally detected by the release of a label (e.g. radioactive substrates, fluorescent dyes or natural intracellular proteins) from the lysed cells. Exemplary effector cells for such assays include peripheral blood mononuclear cells (PBMC) and NK cells.

As example, in an exemplary assay for ADCC activity of an anti-CD33 antibody, CD33-expressing cells may be labeled with ⁵¹Cr and washed extensively. The anti-CD33 antibodies may be added to the CD33-expressing cells at various concentrations, and the assay started by adding effector cells (NK cells from peripheral blood mononuclear cells, for example). After incubation for various time intervals at 37° C., assays are stopped by centrifugation and ⁵¹Cr release from lysed cells is measured in a scintillation counter. The percentage of cellular cytotoxicity may be calculated as the percent maximal lysis which may be induced by adding 3% perchloric acid to the CD33-expressing cells.

In an exemplary assay for cytotoxicity, tetrazolium salt may be added to the CD33-expressing cells treated with various amounts of the anti-CD33 antibody. In living mitochondria, the XTT is reduced to an orange product by mitochondrial dehydrogenase and transferred to the cell surface. The orange product can be optically quantified and reflects the number of living cells. Alternatively, esterases from living cells are known to hydrolyze the colorless calcenin into as fluorescent molecule. The fluorescence can be measured and quantified, and reflects the number of living cells in the sample. The total amount of dead cells may be measured using propidium iodide, which is excluded from live cells by intact membranes. The fluorescence due to the propidium iodide in dead cells may be quantified by flow-cytometry.

In order to assess CDC, complement protein may need to be included in an assay for cytotoxicity. Measurement of apoptosis induction does not require addition of NK cells or complement protein in an assay for cytotoxicity.

As discussed above, treatment of certain cancers with monoclonal antibodies against CD33 has met with varied success. In initial studies with an unconjugated murine anti-CD33 antibody (M195), a few patients had transient decreases in peripheral blast counts at a saturating or supra-saturating dose. Subsequent studies employed a humanized version of M195, lintuzumab (HuM195), which had greater than 8-fold higher binding avidity than M195 and, unlike M195, demonstrated antibody-dependent cell-mediated cytotoxicity (ADCC). Still, while limited studies pointed toward some activity in acute promyelocytic leukemias (APL) when used in in patients with minimal residual disease, lintuzumab has very modest activity as a single agent in AML even at supra-saturating doses that fully blocked CD33 binding sites throughout a 4-week period, with the infrequent achievement of complete or partial remissions limited to patients with low tumor burden. Efficacy can perhaps be increased if supra-saturating doses are given repeatedly, as suggested by a small trial in which very high doses of lintuzumab were given weekly for 5 weeks and then every other week for patients with clinical benefit.

One approach to improve the effectiveness of the immunotherapeutic agent (i.e., anti-CD33 antibody) includes use of a multi-specific antibody. Thus, according to certain aspects of the presently disclosed invention, the methods may comprise administration of an immunotherapy, wherein the immunotherapy may comprise a multi-specific antibody against a first epitope of CD33 and a second epitope of CD33, or against an epitope of CD33 and epitopes of one or more additional different antigens. Thus, the immunotherapy may comprise a multi-specific antibody comprising at least a first target recognition component which specifically binds to an epitope of CD33 and a second target recognition component which specifically binds to an epitope of an antigen other than CD33.

The additional different antigens may be antigens differentially expressed on cells involved in hematological diseases or disorders, and/or cells involved in solid tumors. For example, the additional different antigens may be selected from the group comprising mesothelin, TSHR, CDI19, CD123, CD22, CD30, CD45, CD171, CD138, CS-1, CLL-1, GD2, GD3, B-cell maturation antigen (BCMA), Tn Ag, prostate specific membrane antigen (PSMA), ROR1, FLT3, FAP, TAG72, CD38, CD44v6, CEA, EPCAM, B7H3, KIT, IL-13Ra2, interleukin-11 receptor a (IL-1 IRa), PSCA, PRSS21, VEGFR2, LewisY, CD24, platelet-derived growth factor receptor-beta (PDGFR-beta), SSEA-4, CD20, Folate receptor alpha (FRa), ERBB2 (Her2/neu), MUC, epidermal growth factor receptor (EGFR), EGFRvIII, NCAM, Prostase, PAP, ELF2M, Ephrin B2, IGF-I receptor, CAIX, LMP2, gplOO, bcr-abl, tyrosinase, EphA2, Fucosyl GM1, sLe, GM3, TGS, HMWMAA, o-acetyl-GD2, Folate receptor beta, TEM1/CD248, TEM7R, CLDN6, GPRC5D, CXORF61, CD97, CD 179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6,E7, MAGE A1, MAGEA3, MAGEA3/A6, ETV6-AML, sperm protein 17, XAGEI, Tie 2, MAD-CT-1, MAD-CT-2, Fos-related antigen 1, prostein, survivin and telomerase, PCTA-1/Galectin 8, KRAS, MelanA/MART1, Ras mutant, hTERT, sarcoma translocation breakpoints, ML-IAP, ERG (TMPRSS2 ETS fusion gene), NA17, PAX3, Androgen receptor, Cyclin B 1, MYCN, RhoC, TRP-2, CYP1B 1, BORIS, SART3, PAX5, OY-TES 1, LCK, AKAP-4, SSX2, RAGE-1, human telomerase reverse transcriptase, RU, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, GPA7, and IGLL1.

The first target recognition component may comprise one of: a first full length heavy chain and a first full length light chain, a first Fab fragment, or a first single-chain variable fragment (scFvs). Moreover, the first target recognition component may be derived from lintuzumab (HuM195), gemtuzumab, or vadastuximab. The second target recognition component may comprise one of: a second full length heavy chain and a second full length light chain, a second Fab fragment, or a second single-chain variable fragment (scFvs). Moreover, the second target recognition component may be derived from any of the additional different antigens listed above.

The multi-specific antibody may be a recombinant antibody, a monoclonal antibody, a chimeric antibody, a humanized antibody, a human antibody, or an antibody fragment.

Alternatively, the presently disclosed invention contemplates methods which include administration of a first antibody against at least one antigen of CD33, and administration of a second antibody, wherein the second antibody is against a different epitope of CD33 than the first antibody, or is against an epitope of a different antigen, such as an antigen selected from the list presented above.

Another approach to improve the therapeutic effect of the monoclonal antibodies used in the present methods includes labeling the antibodies with a radionuclide. On treatment of a patient with the radionuclide labelled monoclonal antibody against CD33, the radionuclide may become localized to cells expressing CD33, respectively, such as at a tumor site, and kill those tumor cells. Thus, the presently disclosed invention relates to methods of treating a proliferative disorder comprising administering an effective amount of a monoclonal antibody against CD33 and an effective amount of a chemotherapeutic treatment regime, wherein the antibody is labeled with an effector molecule such as a radionuclide. According to certain aspects of the presently disclosed invention, the monoclonal antibody against CD33 may be lintuzumab (HuM195), gemtuzumab, and/or vadastuximab.

According to certain aspects of the presently disclosed invention, the radiolabeled anti-CD33 targeting agent is radiolabeled HuM195 (lintuzumab). Radiolabeled antibodies envisioned in this invention include, without limitation, ¹³¹I-HuM195, ¹²⁵I-HuM195, ¹²³I-HuM195, ⁹⁰Y-HuM195, ¹⁷⁷Lu-HuM195, ¹⁸⁶Re-HuM195, ¹⁸⁸Re-HuM195, ⁸⁹Sr-HuM195, ¹⁵³Sm-HuM195, ³²P-HuM195, ²²⁵Ac-HuM195, ²¹³Bi-HuM195, ²¹³Po-HuM195, ²¹¹At-HuM195, ²¹²Bi-HuM195, ²¹³Bi-HuM195, ²²³Ra-HuM195, ²²⁷Th-HuM195, ¹⁴⁹Tb-HuM195, ¹³¹I-HuM195, ¹³⁷Cs-HuM195, ²¹²Pb-HuM195 and ¹⁰³Pd-HuM195. Preferably, the radiolabeled anti-CD33 antibody is ¹³¹I-HuM195 or ²²⁵Ac-HuM195.

According to certain aspects of the presently disclosed invention, the targeting agent may be labelled with ¹³¹I or ²²⁵Ac, and may be at least 5-fold more effective at causing cell death of lymphoblast or myeloma cells than a control monoclonal antibody, wherein the control targeting agent comprises an un-labeled targeting agent against the same epitope of the antigen as the ¹³¹I or ²²⁵Ac labelled targeting agent. For example, a ¹³¹I or ²²⁵Ac labelled monoclonal antibody may be at least 10-fold more effective, at least 20-fold more effective, at least 50-fold more effective, or at least 100-fold more effective at causing cell death of lymphoblast or myeloma cells than the control monoclonal antibody.

According to certain aspects of the presently disclosed invention, the methods may comprise administration of labeled and un-labelled (e.g., “naked”) fractions of the anti-CD33 targeting agent, such as an antibody, antibody fragment, etc. For example, the un-labeled fraction may comprise the same antibody against the same epitope as the labeled fraction. In this way, the total radioactivity of the antibody may be varied or may be held constant while the overall antibody protein concentration may held constant or may be varied, respectively. For example, the total protein concentration of un-labelled antibody fraction administered may vary depending on the exact nature of the disease to be treated, age and weight of the patient, identity of the monoclonal antibody, and the label (e.g., radionuclide) selected for labeling of the monoclonal antibody.

According to certain aspects of the presently disclosed invention, the anti-CD33 targeting agent may be ¹³¹I-HuM195 administered at a radiation dose of 10 mCi to 1,200 mCi, as detailed hereinabove. According to certain aspects of the presently disclosed invention, the anti-CD33 targeting agent may be ¹³¹I-labeled anti-CD33 antibody may be administered as one or more doses according to any of the dosing schedules listed herein until the subject has received a cumulative radiation dose of 10 mCi to 300 mCi, such as 20 mCi to 200 mCi, or 20 mCi to 100 mCi.

According to certain aspects of the presently disclosed invention, the anti-CD33 targeting agent may be ²²⁵Ac-HuM195 administered at a radiation dose of less than 5.0 μCi/kg (i.e., where the amount of ²²⁵Ac-labeled antibody administered to the subject delivers a radiation dose of below 5.0 μCi per kilogram of subject's body weight), as detailed hereinabove. According to certain aspects of the presently disclosed invention, the ²²⁵Ac-labeled anti-CD33 antibody may be administered as one or more doses according to any of the dosing schedules listed herein until the subject has received a cumulative radiation dose of 1 μCi/kg, 2 μCi/kg, 3 μCi/kg, 4 μCi/kg, 5 μCi/kg, 6 μCi/kg, 7 μCi/kg, 8 μCi/kg, 9 μCi/kg, or 10 μCi/kg of patient weight. According to certain aspects of the presently disclosed invention, an effective amount of the anti-CD33 antibody may comprises a radiation dose of 0.5 to 4 μCi/kg of subject's body weight.

In general, the effective amount of the anti-CD33 antibody comprises a protein dose of less than 16 mg/kg body weight, such as from 0.01 mg/kg to 10 mg/kg, or 0.01 mg/kg patient weight to 1.0 mg/kg, or 0.01 mg/kg patient weight to 0.1 mg/kg.

According to certain aspects of the presently disclosed invention, the effective amount of the anti-CD33 antibody is a maximum tolerated dose (MTD) of the anti-CD33 antibody.

According to certain aspects of the presently disclosed invention, the radiolabeled monoclonal antibody may exhibit essentially the same immunoreactivity to the antigen as a control monoclonal antibody, wherein the control monoclonal antibody comprises an un-labeled monoclonal antibody against the same epitope of the antigen as the radiolabeled monoclonal antibody.

In an exemplary embodiment of the presently disclosed invention, the anti-CD33 antibody may be lintuzumab conjugated with a radiolabel such as ²²⁵Ac. While the present formulations provide for improved stability of the radiolabeled antibody, i.e., such as maintaining stability of the antibody for at least 24 hours (see WO 2017/155937), the radioisotope eventually degrades the antibody. Thus, an additional advantage of conjugating the anti-CD33 targeting agent with the radionuclides or radiotherapeutic agents disclosed herein is that such an agent may decay the antibody after it has reached the target cells (cells expressing the CD33 antigen) but before it can exert damage to normal cells.

The methods of the presently disclosed invention further include administration of a combination of two or more individual chemotherapeutic agents with the immunotherapeutic agent; and administration of a combination of two or more individual chemotherapeutic agents with the immunotherapeutic agent. As such, the presently disclosed invention contemplates methods of treating a proliferative disease or disorder which includes administration of antibodies against CD33 and administration of two or more individual chemotherapeutic agents such two or more of cladribine, cytarabine, mitoxantrone, and granulocyte colony stimulating factor or filgrastim. According to certain aspects, the methods of the presently disclosed invention include administration of antibodies against CD33 and administration of three or more individual chemotherapeutic agents such three or more of cladribine, cytarabine, mitoxantrone, and granulocyte colony stimulating factor or filgrastim. The chemotherapeutic agents may be delivered together and/or individually during a treatment period, such as on a daily or hourly schedule specific for each chemotherapeutic agent.

The presently disclosed invention further contemplates methods of treating a proliferative disease or disorder which includes administration of antibodies against CD33 and administration of a chemotherapeutic regime, such as CLAG-M. Such a combination of an immunotherapeutic agent and a chemotherapeutic regime may provide a therapeutic effect which is comparable to the effectiveness of a monotherapy, while reducing adverse side effects of the monotherapies, and/or has an improved effectiveness, which may be measured by reduction in the total tumor cell number, increase in the length of time to relapse, and other indicia of patient health; e.g., may provide a synergistic effect over a monotherapy (i.e., either the immunotherapeutic agent or a chemotherapeutic regime).

The chemotherapeutic regime CLAG-M includes administration of a combination of chemotherapeutic agents—cladribine (Leustatin®), filgrastim (Neupogen®), cytarabine (Cytosar-U®), and mitoxantrone (Novantrone®).

Cladribine is an antineoplastic, antimetabolite and purine antagonist. Cladribine is structurally related to fludarabine and pentostatin but has a different mechanism of action. Although the exact mechanism of action has not been fully determined, evidence shows that cladribine is phosphorylated by deoxycytidine kinase to the nucleotidecladribine triphosphate (CdATP; 2-chloro-2′-deoxyadenosine 5′-triphosphate), which accumulates and is incorporated into DNA in cells such as lymphocytes that contain high levels of deoxycytidine kinase and low levels of deoxynucleotidase, resulting in DNA strand breakage and inhibition of DNA synthesis and repair. High levels of CdATP also appear to inhibit ribonucleotide reductase, which leads to an imbalance in triphosphorylateddeoxynucleotide (dNTP) pools and subsequent DNA strand breaks, inhibition of DNA synthesis and repair, nicotinamide adenine dinucleotide (NAD) and ATP depletion, and cell death. Unlike other antimetabolite drugs, cladribine has cytotoxic effects on resting as well as proliferating lymphocytes. However, it does cause cells to accumulate at the G1/S phase junction, suggesting that cytotoxicity is associated with events critical to cell entry into S phase. It also binds purine nucleoside phosphorylase (PNP), however no relationship between this binding and a mechanism of action has been established. Cladribine (2-CdA) may be administered at 3-7 mg per m² body surface area of the subject per day, such as 5 mg per m² body surface area of the subject per day.

Cytarabine is an antimetabolite antineoplastic agent. Cytarabine acts through direct DNA damage and incorporation into DNA. Cytarabine is cytotoxic to a wide variety of proliferating mammalian cells in culture. It exhibits cell phase specificity, primarily killing cells undergoing DNA synthesis (S-phase) and under certain conditions blocking the progression of cells from the G1 phase to the S-phase. Although the mechanism of action is not completely understood, it appears that cytarabine acts through the inhibition of DNA polymerase. A limited, but significant, incorporation of cytarabine into both DNA and RNA has also been reported. Cytarabine (Ara-C) may be administered at 1-3 g per m² body surface area of the subject per day, such as 2 g per m² body surface area of the subject per day.

Mitoxantrone is an anthracenedione-derived antineoplastic agent. Mitoxantrone, a DNA-reactive agent that intercalates into deoxyribonucleic acid (DNA) through hydrogen bonding, causes crosslinks and strand breaks. Mitoxantrone also interferes with ribonucleic acid (RNA) and is a potent inhibitor of topoisomerase IL, an enzyme responsible for uncoiling and repairing damaged DNA. It has a cytocidal effect on both proliferating and non-proliferating cultured human cells, suggesting lack of cell cycle phase specificity. Mitoxantrone may be administered at 5-15 mg per m² body surface area of the subject per day, such as 10 mg per m² body surface area of the subject per day.

Filgrastim is a recombinant, non-glycosylated form of the 175 amino acid protein, granulocyte colony stimulating factor (G-CSF) that induces the proliferation and maturation of neutrophils. Filgrastim binds to the G-CSF receptor and stimulates the production of neutrophils in the bone marrow. As a G-CSF analog, it controls proliferation of committed progenitor cells and influences their maturation into mature neutrophils. Filgrastim also stimulates the release of neutrophils from bone marrow storage pools and reduces their maturation time. Filgrastim acts to increase the phagocytic activity of mature neutrophils. In patients receiving cytotoxic chemotherapy, Filgrastim can accelerate neutrophil recovery, leading to a reduction in duration of the neutropenic phase. Filgrastim may be administered at 100-500 ug filgrastim per day, such as 300 ug filgrastim per day.

According to certain aspects of the presently disclosed invention, the cladribine, may be administered intravenously, the cytarabine may be administered intravenously, the mitoxantrone may be administered intravenously, and the filgrastim may be administered subcutaneously.

According to certain aspects of the methods of the presently disclosed invention, the anti-CD33 antibody and the chemotherapeutic treatment regime may be administered at the same time. As such, they may be provided in a single composition, or may be provided as several different compositions that are combined prior to administration, or may be administered within a similar short time-span (e.g., within minutes or hours of each other). Alternatively, the anti-CD33 antibody and the chemotherapeutic treatment regime may be administered sequentially. As such, the anti-CD33 antibody may be administered before the chemotherapeutic treatment regime, after the chemotherapeutic treatment regime, or both before and after the chemotherapeutic treatment regime. Moreover, the chemotherapeutic treatment regime may be administered before the anti-CD33 antibody, after the anti-CD33 antibody, or both before and after the anti-CD33 antibody.

According to certain aspects of the methods of the presently disclosed invention, the anti-CD33 antibody may be administered according to a dosing schedule selected from the group consisting of once every 5, 7, 10, 12, 14, 20, 21, 24, 28, 30, 35, and 42 days throughout a treatment period, wherein the treatment period includes at least two doses.

According to certain aspects of the presently disclosed invention, the anti-CD33 targeting agent may be administered according to a dose schedule that includes 2 doses, such as on days 1 and 5, 6, 7, 8, 9, or 10 of a treatment period, or days 1 and 8 of a treatment period.

According to certain aspects of the presently disclosed invention, the chemotherapeutic treatment regime may be administered according to a dosing schedule selected from once every 1, 2, 3, 4, 5, or 6 days throughout a treatment period, wherein the treatment period includes at least two doses.

According to certain aspects of the presently disclosed invention, a standard administration protocol for the CLAG-M chemotherapeutic regime includes administration of cladribine intravenously over a time-span of about 2 hours, followed by administration of cytarabine intravenously over a time-span of about 4 hours. The cytarabine administration may be about 2 hours after the cladribine administration. These two agents may be administered daily for 5 days. Additionally, the mitoxantrone may be administered intravenously on days to 3 of the administration protocol. Finally, filgrastim may be administered subcutaneously on days 0 to 5 of the administration protocol.

According to certain aspects of the presently disclosed invention, the immunotherapeutic agent (anti-CD33 antibody) may be administered on any one of days 5 to 30 of the standard administration protocol of the CLAG-M chemotherapeutic regime. For example, the anti-CD33 antibody may be administered on any one of days 9 to 20 of the standard administration protocol of the CLAG-M chemotherapeutic regime, or on days 12, 13, 14, 15, or 16 of the standard administration protocol of the CLAG-M chemotherapeutic regime, or on day 14 of the standard administration protocol of the CLAG-M chemotherapeutic regime.

According to certain aspects of the presently disclosed invention, the methods may comprise administration of labeled and un-labelled (e.g., “naked”) fractions of the monoclonal antibody against CD33. The un-labeled fraction may comprise the same antibody against the same epitope as the labeled fraction. In this way, the total radioactivity of the antibody may be reduced or set while the overall antibody concentration may be varied. For example, the total protein concentration of un-labelled antibody fraction administered may vary depending on the exact nature of the disease to be treated, age and weight of the patient, identity of the monoclonal antibody, and the label (e.g., radionuclide) selected for labeling of the monoclonal antibody.

According to certain aspects of the presently disclosed invention, more than one dose of the immunotherapeutic agent (anti-CD33 antibody) may be administered. Moreover, each dose may be the same, or may be different. For example, a first dose of the immunotherapeutic agent may be larger (induction dose) than additional doses (continuation doses) of the immunotherapeutic agent.

The methods of the presently disclosed invention, which according to certain aspects include administration of monospecific and/or multi-specific immunological agents, and the chemotherapeutic regime CLAG-M, may further comprise administering one or more additional therapeutic agents. The additional therapeutic agents may be relevant for the disease or condition to be treated. Such administration may be simultaneous, separate or sequential with the administration of the effective amount of the immunotherapeutic agent and the CLAG-M chemotherapy regime detailed herein. For simultaneous administration, the agents may be administered as one composition, or as separate compositions, as appropriate.

Exemplary additional therapeutic agents include at least chemotherapeutic agents, anti-inflammatory agents, immunosuppressive agents, immunomodulatory agents, or a combination thereof. Moreover, the one or more further therapeutic agents may comprise an antimyeloma agent, such as dexamethasone, melphalan, doxorubicin, bortezomib, lenalidomide, prednisone, carmustine, etoposide, cisplatin, vincristine, cyclophosphamide, and thalidomide.

Exemplary chemotherapeutic agents include an antimetabolite, such as methotrexate, 6-mercaptopurine, 6-thioguanine, fludarabine, 5-fluorouracil, decarbazine, hydroxyurea, asparaginase, gemcitabine, and similar agents.

Exemplary chemotherapeutic agents include an alkylating agent, such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C, cisplatin and other platinum derivatives, such as carboplatin, and similar agents.

Exemplary chemotherapeutic agents include an antibiotic, such as dactinomycin (formerly actinomycin), bleomycin, calicheamicin, daunorubicin (formerly daunomycin), doxorubicin, idarubicin, mithramycin, mitomycin, mitoxantrone, plicamycin, anthramycin (AMC) and similar agents.

Exemplary chemotherapeutic agents include anti-mitotic agent, such as taxanes, for instance docetaxel, and paclitaxel, and vinca alkaloids, for instance vindesine, vincristine, vinblastine, and vinorelbine.

Exemplary chemotherapeutic agents include a topoisomerase inhibitor, such as topotecan.

Exemplary chemotherapeutic agents include a growth factor inhibitor, such as an inhibitor of ErbB1 (EGFR) (such as gefitinib (Iressa®), cetuximab (Erbitux®), erlotinib (Tarceva®), HuMax-EGFr (2F8 disclosed in WO 2002/100348) and similar agents), an inhibitor of ErbB2 (Her2/neu) (such as trastuzumab (Herceptin®) and similar agents) and similar agents. In one embodiment, such a growth factor inhibitor may be a farnesyl transferase Inhibitor, such as SCH-66336 and R115777. In one embodiment, such a growth factor inhibitor may be a vascular endothelial growth factor (VEGF) inhibitor, such as bevacizumab (Avastin®).

Exemplary chemotherapeutic agents include a tyrosine kinase inhibitor, such as imatinib (Glivec, Gleevec ST1571), lapatinib, PTK787/ZK222584 and similar agents.

Exemplary chemotherapeutic agents include a histone deacetylase inhibitor. Examples of such histone deacetylase inhibitors include hydroxamic acid-based hybrid polar compounds, such as SAHA (suberoylanilide hydroxamic acid).

Exemplary chemotherapeutic agents include a P38a MAP kinase inhibitor, such as SCIO-469.

Exemplary chemotherapeutic agents include inhibitors of angiogenesis, neovascularization, and/or other vascularization. Examples of such inhibitors include urokinase inhibitors, matrix metalloprotease inhibitors (such as marimastat, neovastat, BAY 12-9566, AG 3340, BMS-275291 and similar agents), inhibitors of endothelial cell migration and proliferation (such as TNP-470, squalamine, 2-methoxyestradiol, combretastatins, endostatin, angiostatin, penicillamine, SCH66336 (Schering-Plough Corp, Madison, N.J.), R115777 (Janssen Pharmaceutica, Inc, Titusville, N.J.) and similar agents), antagonists of angiogenic growth factors (such as such as ZD6474, SU6668, antibodies against angiogenic agents and/or their receptors (such as VEGF, bFGF, and angiopoietin-1), thalidomide (Thalomid®), thalidomide analogs (such as CC-5013 (lenalidomide, Revlimid™) and CC4047 (Actimid™), Sugen 5416, SU5402, antiangiogenic ribozyme (such as angiozyme), interferon α (such as interferon α2α), suramin and similar agents), VEGF-R kinase inhibitors and other anti-angiogenic tyrosine kinase inhibitors (such as SU011248), inhibitors of endothelial-specific integrin/survival signaling (such as vitaxin and similar agents), copper antagonists/chelators (such as tetrathiomolybdate, captopril and similar agents), carboxyamido-triazole (CAI), ABT-627, CM101, interleukin-12 (IL-12), IM862, PNU145156E as well as nucleotide molecules inhibiting angiogenesis (such as antisense-VEGF-cDNA, cDNA coding for angiostatin, cDNA coding for p53 and cDNA coding for deficient VEGF receptor-2) and similar agents.

Other examples of such inhibitors of angiogenesis, neovascularization, and/or other vascularization are anti-angiogenic heparin derivatives and related molecules (e.g., heperinase III), temozolomide, NK4, macrophage migration inhibitory factor (MIF), cyclooxygenase-2 inhibitors, inhibitors of hypoxia-inducible factor 1, anti-angiogenic soy isoflavones, oltipraz, fumagillin and analogs thereof, somatostatin analogues, pentosan polysulfate, tecogalan sodium, dalteparin, tumstatin, thrombospondin, NM-3, combrestatin, canstatin, avastatin, antibodies against other relevant targets (such as anti-alpha-v/beta-3 integrin and anti-kininostatin mAbs) and similar agents.

Exemplary chemotherapeutic agents include thalidomide (Thalomid®), thalidomide analogs (such as CC-5013 (lenalidomide, Revlimid™) and/or CC4047 (Actimid™).

Exemplary chemotherapeutic agents may include additional antibody therapeutics, or drugs such as a proteasome inhibitor, such as bortezomib (Velcade®), a corticosteroid, such as prednisone, prednisolone, dexamethasone, etc, a bisphosphonate. Examples of potentially suitable biphosphonates are pamidronate (Aredia®), zoledronic acid (Zometa®), clodronate (Bonefos®), risendronate (Actonel®), ibandronate (Boniva®), etidronate (Didronel®), alendronate (Fosamax®), tiludronate (Skelid®), incadronate (Yamanouchi Pharmaceutical) and minodronate (YM529, Yamanouchi).

Exemplary chemotherapeutic agents include an erythropoietic agent. Examples of suitable erythropoietic agents are erythropoietin (EPO), such as epoetin alfa (for instance Procrit®, Epogen®, and Eprex®) and epoetin beta (for instance NeoRecormon®) and erythropoiesis-stimulating proteins (for instance Aranesp®).

Exemplary chemotherapeutic agents include an anti-anergic agents (for instance small molecule compounds, proteins, glycoproteins, or antibodies that break tolerance to tumor and cancer antigens).

Exemplary chemotherapeutic agents include a virus, viral proteins, and the like. Replication-deficient viruses, that generally are capable of one or only a few rounds of replication in vivo, and that are targeted to tumor cells, may for instance be useful components of such compositions and methods. Such viral agents may comprise or be associated with nucleic acids encoding immunostimulants, such as GM-CSF and/or IL-2. Both naturally oncolytic and such recombinant oncolytic viruses (for instance HSV-1 viruses, reoviruses, replication-deficient and replication-sensitive adenovirus, etc.) may be useful components of such methods and compositions.

Exemplary anti-inflammatory agents may be selected from a steroidal drug and a NSAID (nonsteroidal anti-inflammatory drug). Other anti-inflammatory agents may be selected from aspirin and other salicylates, Cox-2 inhibitors (such as rofecoxib and celecoxib), NSAIDs (such as ibuprofen, fenoprofen, naproxen, sulindac, diclofenac, piroxicam, ketoprofen, diflunisal, nabumetone, etodolac, oxaprozin, and indomethacin), anti-IL6R antibodies, anti-IL8 antibodies, anti-IL15 antibodies, anti-IL15R antibodies, anti-CD4 antibodies, anti-CD11a antibodies (e.g., efalizumab), anti-alpha4/beta-1 integrin (VLA4) antibodies (e.g natalizumab), CTLA4-1 g for the treatment of inflammatory diseases, prednisolone, prednisone, disease modifying antirheumatic drugs (DMARDs) such as methotrexate, hydroxychloroquine, sulfasalazine, pyrimidine synthesis inhibitors (such as leflunomide), IL-1 receptor blocking agents (such as anakinra), TNF-α blocking agents (such as etanercept, infliximab, and adalimumab) and similar agents.

Exemplary immunosuppressive and/or immunomodulatory agents include cyclosporine, azathioprine, mycophenolic acid, mycophenolate mofetil, corticosteroids such as prednisone, methotrexate, gold salts, sulfasalazine, antimalarials, brequinar, leflunomide, mizoribine, 15-deoxyspergualine, 6-mercaptopurine, cyclophosphamide, rapamycin, tacrolimus (FK-506), OKT3, anti-thymocyte globulin, thymopentin, thymosin-α and similar agents.

According to certain aspects of the presently disclosed invention, the additional therapeutic agents may comprise an antimyeloma agent. Exemplary antimyeloma agents include dexamethasone, melphalan, doxorubicin, bortezomib, lenalidomide, prednisone, carmustine, etoposide, cisplatin, vincristine, cyclophosphamide, and thalidomide, several of which are indicated above as chemotherapeutic agents, anti-inflammatory agents, or immunosuppressive agents.

According to certain aspects of the presently disclosed invention, the additional therapeutic agents may comprise allopurinol, administered at a dose of 300-600 mg/day orally starting on day 1 of the treatment period and continuing for at least 7 days after the CD33 targeting agent. Prophylactic antibiotics and antifungal therapies may be included for those patients who have an absolute neutrophil count of less than 500/ul. Analgesics and antihistamines may also be included prior at administration of the CD33 targeting agent by infusion to reduce infusion-related reactions.

The additional therapeutic agents may be administered according to any standard dose regime known in the field. For example, therapeutic agents may be administered at concentrations in the range of 1 to 500 mg/m², the amounts being calculated as a function of patient surface area (m²). For example, exemplary doses of paclitaxel may include 15 mg/m2 to 275 mg/m², exemplary doses of docetaxel may include 60 mg/M2 to 100 mg/m², exemplary doses of epithilone may include 10 mg/M2 to 20 mg/m², and an exemplary dose of calicheamicin may include 1 mg/m² to 10 mg/m². While exemplary doses are listed herein, such are only provided for reference and are not intended to limit the dose ranges of the drug agents of the presently disclosed invention.

The methods of the presently disclosed invention may further comprise transplanting autologous or allogeneic stem cells to the subject after administration of the anti-CD33 antibody. When the anti-CD33 antibody is labelled with a radionuclide, the stem cells may be transplanted at a time after administration of the anti-CD33 antibody when a radiation dose from the anti-CD33 antibody is not harmful to the transplanted cells, such as 8 to 20 days after the administration of the anti-CD33 antibody, or even 10 to 16 days after administration of the anti-CD33 antibody.

The presently disclosed invention provides methods for treating a subject having a proliferative disorder, for inhibiting growth and/or proliferation of a cell expressing CD33, and for treating a disease or disorder involving cells expressing CD33. The proliferative disorder may be a hematological malignancy.

Thus, the presently disclosed invention provides methods for treating a subject having a proliferative disorder. In certain instances, the proliferative disorder may be a hematological hematological malignancy. According to certain aspects of the presently disclosed invention, the hematological malignancy may be one or more of multiple myeloma, acute myeloid leukemia, myelodysplastic syndrome, and myeloproliferative neoplasm. According to certain aspects of the invention, the cell expressing CD33 may be a multiple myeloma cell.

According to certain aspects of the presently disclosed invention, methods are provided for treating a solid cancer in a subject, wherein the method comprises administering to the subject a therapeutically effective amount of the monoclonal antibodies detailed herein (i.e., monotherapy with anti-CD33 antibodies, and/or a bi-specific antibody against epitopes of CD33 and another antigen as detailed above), and the chemotherapeutic regime CLAG-M. Without wishing to be bound by any particular theory, based on the immunomodulatory effects observed with the anti-CD33 monoclonal antibody described herein, the methods of the presently disclosed invention may be efficacious in treatment of solid tumors. The treatment may include intravenous, intraperitoneal, or intra-tumoral infusion of the immunotherapeutic agents.

The following aspects are disclosed in this application:

Aspect 1. A method for inhibiting growth and/or proliferation of a cell expressing CD33, the method comprising: administering to a subject an effective amount of an anti-CD33 targeting agent and an effective amount of a combination of two or more individual chemotherapeutic agents.

Aspect 2. The method of aspect 1, wherein the combination of two or more individual chemotherapeutic agents comprises two or more of cladribine, cytarabine, mitoxantrone, and filgrastim.

Aspect 3. The method according to any one of aspects 1 or 2, wherein the combination of two or more individual chemotherapeutic agents comprises two or more of: cladribine, administered intravenously; cytarabine, administered intravenously; mitoxantrone, administered intravenously; and filgrastim, administered subcutaneously.

Aspect 4. The method according to any one of aspects 1 to 3, wherein the effective amount of the combination of two or more individual chemotherapeutic agents comprises two or more of: 3-7 mg cladribine per m² body surface area of the subject per day; 1-3 g cytarabine per m² body surface area of the subject per day; 5-15 mg mitoxantrone per m² body surface area of the subject per day; and 100-500 ug filgrastim per day.

Aspect 5. The method according to any one of aspects 1 to 4, wherein the effective amount of the combination of two or more individual chemotherapeutic agents comprises: 5 mg cladribine per m² body surface area of the subject per day; 2 g cytarabine per m² body surface area of the subject per day; 10 mg mitoxantrone per m² body surface area of the subject per day; and 300 ug filgrastim per day.

Aspect 6. The method according to any one of aspects 2 to 5, wherein the effective amount of the combination is administered to the subject as: cladribine on days 1, 2, 3, 4, and 5 of a treatment period; cytarabine on days 1, 2, 3, 4, and 5 of the treatment period; mitoxantrone on days 1, 2, and 3 of the treatment period; and filgrastim, on days 0, 1, 2, 3, 4, and 5 of the treatment period.

Aspect 7. The method according to any one of aspects 1 to 6, wherein the anti-CD33 targeting agent is administered on any one of day 1 to 30 of a treatment period; or on any one of day 9 to 20 of a treatment period; or on day 12, 13, 14, 15, or 16 of the treatment period; or on day 14 of the treatment period.

Aspect 8. The method according to any one of aspects 1 to 7, wherein the anti-CD33 targeting agent and the chemotherapeutic agent are administered sequentially.

Aspect 9. The method according to any one of aspects 1 to 8, wherein the anti-CD33 targeting agent is administered before the chemotherapeutic agent; or wherein the anti-CD33 targeting agent is administered after the chemotherapeutic agent.

Aspect 10. The method according to any one of aspects 1 to 9, wherein the anti-CD33 targeting agent comprises a radiolabel antibody against CD33, wherein the radiolabel is selected from ¹³¹I, ¹²⁵I, ¹²³I, ⁹⁰Y, ¹⁷⁷Lu, ¹⁸⁶Re, ¹⁸⁸Re, ⁸⁹Sr, ¹⁵³Sm, ³²P, ²²⁵Ac, ²¹³Bi, ²¹³Po, ²¹¹At, ²¹²Bi, ²¹³Bi, ²²³Ra, ²²⁷Th, ¹⁴⁹Tb, ¹³⁷Cs, ²¹²Pb or ¹⁰³Pd, or a combination thereof.

Aspect 11. The method according to any one of aspects 1 to 10, wherein the anti-CD33 targeting agent comprises lintuzumab, gemtuzumab, vadastuximab, or a combination thereof.

Aspect 12. The method according to any one of aspects 1 to 11, wherein the anti-CD33 targeting agent comprises HuM195.

Aspect 13. The method according to any one of aspects 1 to 12, wherein the anti-CD33 targeting agent is ²²⁵Ac-labelled and the effective amount of the anti-CD33 targeting agent comprises a radiation dose of 0.1 to 10 uCi/kg body weight of the subject, or 0.2 to 8 uCi/kg body weight of the subject, or 0.5 to 4 uCi/kg subject body weight, or 0.2 to 2 uCi/kg subject body weight.

Aspect 14. The method according to any one of aspects 1 to 13, wherein the anti-CD33 targeting agent is ¹³¹I-labelled and the effective amount of the anti-CD33 targeting agent comprises a radiation dose 10 mCi to 200 mCi, or 50 mCi to 150 mCi, or 50 mCi to 100 mCi.

Aspect 15. The method according to any one of aspects 1 to 14, wherein the effective amount of the anti-CD33 targeting agent comprises a protein dose of less than 16 mg/kg body weight of the subject, less than 10 mg/kg body weight of the subject, or less than 6 mg/kg body weight of the subject, less than 500 μg/kg body weight of the subject, less than 100 μg/kg body weight of the subject, less than 50 μg/kg body weight of the subject, or less than 20 μg/kg body weight of the subject.

Aspect 16. The method according to any one of aspects 1 to 15, wherein the method may treat a hematological disease or disorder selected from one or more of acute lymphoid leukemia, acute myeloid leukemia, chronic myelomonocytic leukemia, an erythrocytic leukemia, acute megakaryoblastic leukemia, histiocytic lymphoma, a myeloid sarcoma, a mast cell proliferative disorder, and myelodysplastic syndrome.

Aspect 17. The method according to any one of aspects 1 to 16, wherein the method may treat a relapsed/refractory hematological disease or disorder, wherein the hematological disease or disorder is selected from multiple myeloma, acute myeloid leukemia, myelodysplastic syndrome, and myeloproliferative neoplasm.

Aspect 18. The method according to any one of aspects 1 to 17, wherein the cells expressing CD33 are blast cells, such as myeloblast cells or malignant plasmacytes.

### EXAMPLES

### Example 1: CD33 Specificity and Stability

Lintuzumab conjugated with Actinium-225 (Ac²²⁵) was tested for cytotoxicity against specific cell types which express CD33. For example, suspensions of HL60 (leukemia cells) were incubated with various doses of radiolabeled lintuzumab (lintuzumab-Ac²²⁵), and the dose at which 50% of the cells were killed (LD₅₀) was found to be 8 μCi per mL of cell suspension.

In studies to access the reactivity of the radiolabeled lintuzumab with peripheral blood and bone marrow cells from nonhuman primate and human frozen tissues, the radiolabeled lintuzumab showed reactivity with mononuclear cells only, demonstrating specificity. Moreover, in studies to determine the stability of the radiolabel on the antibody, 10 normal mice (8 week old Balb/c female mice from Taconic, Germantown, N.Y.) were injected in the tail with 300 nCi radiolabeled lintuzumab (in 0.12 ml). Serum samples taken over a 5 day period showed that the Actinium-225 remained bound to the lintuzumab, demonstrating the stability of the radiolabel on the antibody in vivo.

A maximum tolerated dose (MTD) of a single injection of the radiolabeled lintuzumab was determined to be 3 uCi/kg patient weight. As a split dose (e.g., 2 equal doses administered 4-7 days apart), the MTD was determined to be 2 uCi/kg per dose, or 4 uCi/kg total. This data was determined by injections into patients with relapsed/refractory AML: 21 patients were injected with increasing doses of the radiolabeled lintuzumab—0.5 uCi/kg to 4 uCi/kg. Determination of MTD was based on the severity of the adverse effects observed at each dose level. Anti-leukemic effects included elimination of peripheral blood blasts in 13 of 19 evaluable patients. Twelve of 18 patients who were evaluable at 4 weeks following treatment had reductions in bone marrow blasts, including nine with reductions ≥50%. Three patients treated with 1 uCi/kg, 3 uCi/kg and 4 uCi/kg respectively had ≤5% blasts after therapy.

### Example 2: CD33 Human Maximal Tolerated Dose and Efficacy

A Phase I trial will be used to determine the MTD of fractionated doses of lintuzumab-Ac²²⁵ followed by Granulocyte Colony Stimulating factor (GCSF) support in each cycle. A cycle in general is approximately 42 days. A cycle starts with administration of a fractionated dose of Lintuzumab-Ac²²⁵ on Day 1 followed by the administration of GCSF on Day 9 and continuing GCSF per appropriate dosing instructions until absolute neutrophil count (ANC) is greater than 1,000, which is expected to occur within 5-10 days. On Days 14, 21, 28, 35 and 42 peripheral blood will be assessed for paraprotein burden. A bone marrow aspirate will be performed to assess plasmocyte infiltration on Day 42. If a response is a partial response or better but less than a complete response on Day 42, and the patient remains otherwise eligible, the patient will be re-dosed in a new cycle at the same dose level no sooner than 60 days after Day 1 of the first cycle. In absence of dose limiting toxicities, cycles will continue using the above described algorithm until the patient has received a cumulative dose of 4 μCi/kg of lintuzumab-Ac²²⁵.

### Example 3: CD33 Maximal Tolerated Dose when Added with CLAG-M

The CLAG-M treatment regimen consists of the following agents Cladribine 5 mg/M2 IV over two hours on days 1-5; Cytarabine 2 gm/M2 IV over four hours on days 1-5, starting two hours after the Cladribine infusion is complete; Mitoxantrone 10 mg/m2 IV on days 1-3; G-CSF (filgrastim) at a dose of 300 μg on days 0-5. Lintuzumab-Ac²²⁵ will be administered on day 14 of therapy as an IV infusion in 30 minutes. The dose of Lintuzumab-Ac²²⁵ for the three study cohorts is mentioned below. Prophylaxis for radiation induced nephrotoxicity will be done using treatment.

| | | Lintuzumab |
|---|---|---|
| Cohort | Ac²²⁵ | (HuM195) |
| 1 | 0.5 uCi/kg | 15 ug/kg |
| 2 | 1.0 uCi/kg | 15 ug/kg |
| 3 | 1.5 uCi/kg | 15 ug/kg |

Monitoring and response assessment—Bone marrow biopsies will be obtained at baseline (when relapse is diagnosed) and after CLAG-M therapy—up to 42 days after administration of Lintuzumab-Ac²²⁵ or at recovery of counts, whichever is earlier. Patients will be followed with daily complete blood counts and metabolic panel during their hospital stay. Response to therapy will be assessed according to the standard definitions developed by the international working group, with complete remission being bone marrow blasts <5% with absolute neutrophil count ≥1000/L and platelet ≥100,000/L. Complete remission (CR) without platelet recovery will be defined as a CRp and without white blood count recovery will be defined as CRi. Partial remission (PR) is defined by a decrease of at least 50% in the percentage of blasts to 5-25% in the bone marrow and normalized blood counts. Those who do not achieve a CR, Cri, CRp or PR will be defined as non-response (NR) group. Progression-free survival (PFS) will be calculated from the first day of remission until documentation of relapse. Overall survival (OS) will be calculated from the first day of salvage therapy until death. Hematological and extra hematological toxicity will be assessed according to the CTCAE criteria. DLT will include the following events unless they could be attributed to an alternative cause other than the treatment.

### Example 4: CD33 Combination Therapy with a Chemotherapeutic Agent

In a phase 1 clinical trial, eighteen patients with relapsed AML were treated with ²²⁵Ac-linutuzmab administered in fractionated doses on days 1 and 8 combined with low dose Ara-C (LDAC). Treatment was found to induce remissions in older patients with untreated AML at doses above 0.5 uCi/kg/fraction. In a Phase 2 portion of the clinical trial, thirteen patients with initial presentation of AML who were considered to be unfit for cytotoxic chemotherapy were treated with ²²⁵Ac-linutuzmab administered in fractionated doses on days 1 and 8 without LDAC. Preliminary data are available on 9 with median age 75 years (range, 65-82) and PS median 2 (0-1 in 2 patients, 2 in 3 patients, & 3 in 2 patients). Six (67%) had prior treatment for AHDs (5 MDS, 1 atypical CML). At baseline, 5 patients had ANC≥500/μL, only 2 had ANC≥1000/μL, and only 1 had platelets >50,000/μL.

Myelosuppression was seen in all evaluable patients including grade 4 thrombocytopenia with marrow aplasia for >6 weeks following therapy in 3 patients. The only Grade >3 non-hematologic toxicities reported in ≥1 pt were pneumonia and cellulitis. Veno-occlusive disease did not occur. The 30-day mortality rate was 33% (disease progression, acute on chronic respiratory failure, and post-traumatic intracranial hemorrhage after a fall).

Objective responses were documented in 5 of the 9 patients (56%): 2 Complete Remissions with incomplete platelet count recovery (CRp) and 3 Complete Remissions with incomplete hematologic recovery (CRi). Two patients had resistant disease.

Median time to neutrophil recovery (ANC≥500/μL) was 36 days (range 20-60) from the first dose of ²²⁵Ac-lintuzumab. The two patients with CRp had neutrophil recovery at Days 60 and 36. Two of the patients with CRi had not reached ANC≥500/μL when they expired from infection on days 65 and 56, and the third is at day 66+ without ANC recovery. Since patients with antecedent hematologic disorders (AHDs) may not have capacity to recover to normal neutrophil production, patients without AHDs may be more informative. Of the 3 patients without AHDs, 1 had ANC recovery at Day 36, 1 had death from infection at Day 56 without ANC recovery, and 1 is pending ANC recovery at Day 66+. No patients reached platelet counts >20,000/μL without transfusions.

Preliminary data from this Phase 2 trial of ²²⁵Ac-linutuzmab monotherapy at 2 μCi/kg/fraction document a 56% response rate in older patients unfit for intensive therapy, many with AHDs. As myelosuppression at this dose was considered to be longer than acceptable in this population, accrual to this study will continue at 1.5 μCi/kg/fraction with the goal to shorten recovery times.

### Example 5: CD33 Combination Therapy with CLAG-M

In a phase I clinical trial, patients will be admitted for the administration of CLAG-M chemotherapy. Baseline characteristics such as age, gender, race, AML subtype, date of initial diagnosis, date of relapse, interval between diagnosis and relapse, cytogenetics and molecular mutations at diagnosis, prior cycles of chemotherapy or human stem cell transplantation (HSCT), baseline laboratory tests on admission, percentage of blasts in peripheral blood and bone marrow at diagnosis and relapse will be obtained. The CLAG-M chemotherapy regimen will be administered as described herein. Patients will be followed with daily complete blood counts and metabolic panel during their hospital stay. Bone marrow (BM) study obtained at the time of diagnosis of RR-AML will be used as baseline. Repeat BM study will be obtained after the completion of therapy when absolute neutrophil count (ANC) recovers to >1000 cells/cu·mm or up to 42 days after administration of Lintuzumab-Ac²²⁵ (day+49), whichever is earlier. Response to therapy will be assessed according to the standard definitions developed by the international working group. Patients responding to therapy may be subsequently considered for allogeneic stem cell transplantation. Those with PR or no response could be given additional chemotherapy at the treating physician's discretion and patient's tolerability. Hematological and extra hematological toxicity will be assessed according to National Cancer Institute Common Terminology Criteria for Adverse Events version 4.0.

Dose-escalation will be conducted according to a 3+3 design. CLAG-M chemotherapy will be administered at a fixed dose and schedule. The initial dose of Lintuzumab-AC²²⁵ will be 0.25 uCi/kg (Dose level 1), and the highest dose administered will be 0.75 uCi/kg.

The dose limiting toxicity (DLT) observation period for dose-escalation will be 1 cycle. The first patient at each new dose level must be observed for 1 cycle for occurrence of AEs prior to treating the second patient at dose level. Patients will be entered sequentially to each dose level.

For each dose level, if none of the first 3 patients at that level experiences a DLT, new patients may be entered at the next higher dose level. If 1 of 3 patients experiences a DLT, up to 3 more patients are to be treated at that same dose level. If none of the additional 3 patients at that dose level experiences a DLT, new patients may be entered at the next higher dose level. However, if 1 or more of the additional 3 patients experience a DLT, then no further patients are to be started at that dose level and the preceding dose is the MTD. The MTD will be defined as the highest dose level at which none of the first 3 treated patients, or no more than 1 of the first 6 treated patients, experiences a DLT.

1.-20. (canceled)

21. A method for inhibiting growth and/or proliferation of a cell expressing CD33, the method comprising:
administering to a subject an effective amount of an anti-CD33 targeting agent and an effective amount of a combination of two or more individual chemotherapeutic agents,
wherein the combination of two or more individual chemotherapeutic agents comprises two or more selected from the group consisting of cladribine, cytarabine, mitoxantrone, and granulocyte colony stimulating factor or filgrastim.

22. The method of claim 21, wherein the effective amount of the combination of two or more individual chemotherapeutic agents comprises two or more of:
3-7 mg cladribine per m2 body surface area of the subject per day;
1-3 g cytarabine per m2 body surface area of the subject per day;
5-15 mg mitoxantrone per m2 body surface area of the subject per day; and
100-500 ug filgrastim per day.

23. The method of claim 22, wherein the effective amount of the combination of two or more individual chemotherapeutic agents comprises:
5 mg cladribine per m2 body surface area of the subject per day;
2 g cytarabine per m2 body surface area of the subject per day;
10 mg mitoxantrone per m2 body surface area of the subject per day; and
300 ug filgrastim per day.

24. The method according to claim 22, wherein the effective amount of the combination of two or more individual chemotherapeutic agents is administered to the subject as: cladribine on days 1, 2, 3, 4, and 5 of a treatment period; cytarabine on days 1, 2, 3, 4, and 5 of the treatment period; mitoxantrone on days 1, 2, and 3 of the treatment period; and filgrastim, on days 0, 1, 2, 3, 4, and 5 of the treatment period.

25. The method of claim 21, wherein the anti-CD33 targeting agent is administered on any one of day 5 to 30 of the treatment period.

26. The method of claim 21, wherein the anti-CD33 targeting agent is administered on any one of day 9 to 20 of the treatment period

27. The method of claim 21, wherein the anti-CD33 targeting agent is administered on day 14 of the treatment period 34. (New) The method of claim 32, wherein the radiolabeled BC8 is ²²⁵Ac-BC8, and the effective amount of ²²⁵Ac-BC8 is selected from the group consisting of from 0.1 μCi/kg to 5.0 μCi/kg subject weight, from 0.1 μCi/kg to 1.0 μCi/kg subject weight, from 1.0 μCi/kg to 3.0 μCi/kg subject weight, and from 3.0 μCi/kg to 5.0 μCi/kg subject weight.

28. The method of claim 21, wherein the anti-CD33 targeting agent and the combination of two or more individual chemotherapeutic agents are administered sequentially.

29. The method of claim 21, wherein the anti-CD33 targeting agent comprises a radiolabel antibody against CD33, wherein the radiolabel is selected from ¹³¹I, ¹²⁵I, ¹²³I, ⁹⁰Y, ¹⁷⁷Lu, ¹⁸⁶Re, ¹⁸⁸Re, ⁸⁹Sr, ¹⁵³Sm, ³²P, ²²⁵Ac, ²¹³Bi, ²¹³Po, ²¹¹At, ²¹²Bi, ²¹³Bi, ²²³Ra, ²²⁷Th, ¹⁴⁹Tb, ¹³⁷Cs, ²¹²Pb or ¹⁰³Pd, or a combination thereof.

30. The method of claim 29, wherein the anti-CD33 targeting agent comprises lintuzumab, gemtuzumab, vadastuximab, or a combination thereof.

31. The method of claim 21, wherein the anti-CD33 targeting agent comprises HuM195.

32. The method of claim 31, wherein the anti-CD33 targeting agent is ²²⁵Ac-labelled and the effective amount of the anti-CD33 targeting agent comprises a radiation dose of 0.1 to 10 uCi/kg body weight of the subject.

33. The method of claim 31, wherein the anti-CD33 targeting agent is ¹³¹I-labelled and the effective amount of the anti-CD33 targeting agent comprises a radiation dose 10 mCi to 1200 mCi.

34. The method according to claim 31, wherein the effective amount of the anti-CD33 targeting agent comprises a protein dose of less than 10 mg/kg body weight of the subject.

35. The method of claim 21, wherein the method may treat a hematological disease or disorder selected from one or more of multiple myeloma, acute myeloid leukemia, myelodysplastic syndrome, and myeloproliferative neoplasm.

36. The method of claim 21, wherein the method may treat relapsed/refractory acute myeloid leukemia.

37. The method of claim 21, wherein the cells expressing CD33 are blast cells, such as myeloblast cells or malignant plasmacytes.

38. A method for treating a hematological disease or disorder, the method comprising:
administering to a subject having the hematological disease or disorder an effective amount of a radiolabeled anti-CD33 targeting agent and an effective amount of a chemotherapeutic regime comprising cladribine, cytarabine, mitoxantrone, and filgrastim,
wherein the radiolabeled anti-CD33 targeting agent comprises a composition of ²²⁵Ac-labelled HuM195 and un-labelled HuM195.

39. The method of claim 38, wherein the hematological disease or disorder is relapsed/refractory acute myeloid leukemia.

40. The method of claim 38, wherein the composition comprising ²²⁵Ac-labelled HuM195 and un-labelled HuM195 comprises:
a radiation dose of 0.1 to 10 uCi/kg body weight of the subject; and
a protein dose of less than 16 mg/kg body weight of the subject.


SEQUENCE LISTING (1 sequence)

SEQ ID NO: 1 - protein, 364 aa, Homo sapiens
MPLLLLLPLLWAGALAMDPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ